(19) United States
(12) Patent Application Publication
(10) Pub. No.: US 2018/0271372 A1
(43) Pub. Date: Sep. 27, 2018

(54) MAGNETIC RESONANCE COMPATIBLE ULTRASOUND PROBE

(71) Applicant: General Electric Company, Schenectady, NY (US)

(72) Inventors: Warren Lee, Niskayuna, NY (US); Eric William Fiveland, Niskayuna, NY (US); David Andrew Shoudy, Niskayuna, NY (US); Timothy Fiorillo, Schenectady, NY (US); Kwok Pong Chan, Niskayuna, NY (US); Lowell Scott Smith, Niskayuna, NY (US); James Sabatini, Schenectady, NY (US); David Martin Mills, Niskayuna, NY (US); Thomas Kwok-Fah Foo, Clifton Park, NY (US)

(21) Appl. No.: 15/897,964

(22) Filed: Feb. 15, 2018

Related U.S. Application Data
(60) Provisional application No. 62/477,294, filed on Mar. 27, 2017.

(51) Int. Cl. A61B 5/00; A61B 5/06; A61B 8/00

(52) U.S. Cl. CPC A61B 5/0035; A61B 5/055; A61B 8/4444; A61B 5/066

(57) ABSTRACT

An ultrasound probe configured for use in a multi-modality imaging system includes a body including one or more electrical components of the ultrasound probe, an outermost housing enclosing the ultrasound probe, and an electromagnetic interference (EMI) shield disposed between the body and the housing, wherein the EMI shield is configured to reduce interference between the ultrasound probe and one or more different imaging systems of the multi-modality imaging system. The ultrasound probe further includes a transducer disposed on a patient-facing surface of the ultrasound probe and a cable coupled to the body and configured to communicatively couple the ultrasound probe to an ultrasound imaging system of the multi-modality imaging system, wherein the ultrasound probe comprises substantially non-ferromagnetic material.

### CROSS-REFERENCE TO RELATED APPLICATIONS

This application is claims priority to U.S. Patent Provisional Application No. 62/477,294, entitled “MAGNETIC RESONANCE COMPATIBLE ULTRASOUND PROBE”, filed Mar. 27, 2017, which is herein incorporated by reference in its entirety.

### STATEMENT REGARDING FEDERALLY SPONSORED RESEARCH & DEVELOPMENT

This invention was made with Government support under contract number R01CA190298 awarded by the National Cancer Institute (NCI)/National Institute of Biomedical Imaging and Bioengineering (NIBIB) of the National Institutes of Health (NIH). The Government has certain rights in the invention.

### BACKGROUND

In radiation therapy procedures, along with other therapies and procedures, the ability to manage motion and reduce margins around a tumor or other structure may lead to improved control of diseases, reduced damage to surrounding tissue, and better patient outcomes. In radiation therapy treatment of cancer, specifically, it is important to deliver a radiation dose to the target tumor while avoiding healthy tissue. However, delivery of the radiation dose to the tumor may be complicated by tumor motion due to respiration. Typical methods for motion management include forced shallow breathing, abdominal compression, breath-holds, respiratory gating, and methods of tumor tracking, including implantation of fiducial markers. However, many of these methods may be associated with quality assurance challenges and may not be well tolerated in sick patients. Image-guided radiation therapy (IgRT) procedures can significantly improve the accuracy of radiotherapy treatments by confirming the radiation therapy beam placement at the time of delivery. IgRT systems utilizing magnetic resonance (MR) imaging can provide excellent soft tissue image quality, but a drawback is the relatively low image update rate. Conversely, a strength of ultrasound imaging is the ability to provide real-time volumetric images.

A multi-modality system combining MR and real-time volumetric ultrasound imaging thus has the potential to provide clinicians with the soft-tissue image quality of MR images at the real-time frame rates of ultrasound. However, existing ultrasound probes capable of real-time three-dimensional (3D) imaging are not MR compatible. Furthermore, some ultrasound probes used for IgRT require robotic manipulation to hold the probe in place, which may interfere with treatments.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible embodiments. Indeed, the disclosure may encompass a variety of forms that may be similar to or different from the embodiments set forth below.

In one embodiment, an ultrasound probe configured for use in a multi-modality imaging system, includes a body including one or more electrical components of the ultrasound probe, an outermost housing enclosing the ultrasound probe, and an electromagnetic interference (EMI) shield surrounding the body and disposed between the body and the housing, wherein the first EMI shielding is configured to reduce interference between the ultrasound probe and one or more different imaging systems of the multi-modality imaging system. The ultrasound probe further includes a transducer disposed on a patient-facing surface of the ultrasound probe and a cable coupled to the body and configured to communicatively couple the ultrasound probe to an ultrasound imaging system of the multi-modality imaging system, wherein the ultrasound probe comprises substantially non-ferromagnetic material.

In another embodiment, a multi-modality imaging system includes an ultrasound imaging system, a magnetic resonance (MR) imaging system, wherein the MR imaging system is positioned within a shielded MR room having an MR room shield, an MR-compatible ultrasound probe coupled to the ultrasound imaging system and configured to acquire ultrasound images while the MR-compatible ultrasound probe is positioned within the shielded MR room, wherein all or part of the ultrasound imaging system is positioned outside of the shielded MR room, and a shielded ultrasound probe cable coupled to the MR-compatible ultrasound probe at a first end and coupled to the ultrasound system at a second end.

In another embodiment, a method includes positioning one or more electrical components of an ultrasound probe within a body, surrounding the body with a first electromagnetic interference (EMI) shield, wherein the first EMI shield is configured to reduce interference between the ultrasound probe and one or more different imaging systems, enclosing the body and the first EMI shield within a housing, wherein the first EMI shield is disposed between the body and the housing, and wherein the first EMI shield contacts the housing, disposing a transducer on a patient-facing surface of the ultrasound probe, wherein the transducer includes non-ferromagnetic materials, and coupling a cable to the body, wherein the cable is configured to communicatively couple the ultrasound probe to an ultrasound imaging system.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 illustrates a schematic diagram of an embodiment of a combined magnetic resonance (MR) and ultrasound imaging system, in accordance with aspects of the present disclosure;

FIG. 2 illustrates an embodiment of the combined MR and ultrasound imaging system of FIG. 1 having an MR-compatible ultrasound probe, in accordance with aspects of the present disclosure;

FIG. 3 illustrates an alternative embodiment of the combined MR and ultrasound imaging system of FIG. 1 having a split ultrasound system arrangement, in accordance with aspects of the present disclosure;

FIG. 4 illustrates an embodiment of a connection between the MR-compatible ultrasound probe and an ultrasound imaging system of the combined MR and ultrasound imaging system of FIG. 2, in accordance with aspects of the present disclosure;

FIG. 5 illustrates another embodiment of a connection between the MR-compatible ultrasound probe and the ultrasound imaging system of the combined MR and ultrasound imaging system of FIG. 2, in accordance with aspects of the present disclosure;

FIG. 6 illustrates a flowchart of an embodiment of a pre-treatment method utilizing the combined MR and ultrasound imaging system of FIG. 1, in accordance with aspects of the present disclosure;

FIG. 7 illustrates a flowchart of an embodiment of a treatment method utilizing the combined MR and ultrasound imaging system of FIG. 1, in accordance with aspects of the present disclosure;

FIG. 8 illustrates a perspective view of an embodiment of the MR-compatible ultrasound probe, in accordance with aspects of the present disclosure;

FIG. 9 illustrates a cut-away view of an embodiment of the MR-compatible ultrasound probe of FIG. 8, in accordance with aspects of the present disclosure;

FIG. 10 illustrates a cross-sectional view of an embodiment of the MR-compatible ultrasound probe of FIG. 8, in accordance with aspects of the present disclosure;

FIG. 11A illustrates an example of an MR compatibility test for acoustic stack material using a conventional acoustic stack material;

FIG. 11B illustrates an example of an MR compatibility test for acoustic stack material using and MR-compatible acoustic stack material, in accordance with aspects of the present disclosure;

FIG. 12A illustrates an example of a test of MR compatibility of the MR-compatible ultrasound probe with no probe present;

FIG. 12B illustrates an example of a test of MR compatibility of the MR-compatible ultrasound probe of FIG. 8, in accordance with aspects of the present disclosure;

FIG. 13 illustrates a perspective view of an embodiment of the MR-compatible ultrasound probe of FIG. 8 positioned on a patient, in accordance with aspects of the present disclosure;

FIG. 14A illustrates a cut-away view of an embodiment of a shielded probe cable that may be utilized with the MR-compatible ultrasound probe of FIG. 8, in accordance with aspects of the present disclosure;

FIG. 14B illustrates a cross-sectional view of the shielded probe cable of FIG. 12A, in accordance with aspects of the present disclosure;

FIG. 15A illustrates an example of an ultrasound image obtained using an ultrasound probe and probe cable having incomplete shielding; and

FIG. 15B illustrates an example of an ultrasound image obtained using the MR-compatible ultrasound probe of FIG. 8 and the shielded probe cable of FIGS. 12A and 12B having approximately full shielding, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As used herein, the term “virtual real-time MR image(s)” refers to the display of previously acquired MR images that correspond to a current respiratory state of a patient (as further explained below). Thus, displaying these MR images provides “real-time” MR imaging of the patient even though the current image modality being employed is ultrasound. By displaying the correct previously acquired MR images or set of MR images that accurately represents the positions of the anatomical structures within the imaging field-of-view, a system and process is described that enables real-time viewing of corresponding MR images when another imaging modality, such as ultrasound, is employed.

Combining MR and real-time volumetric ultrasound imaging has the potential to provide clinicians with the soft-tissue image quality of MR images at the real-time frame rates of ultrasound. Existing ultrasound probes capable of real-time three-dimensional (3D) imaging are typically not MR compatible. In particular, MR-compatible ultrasound probes typically only provide two-dimensional (2D) images. Real-time three-dimensional ultrasound imaging can be achieved in two ways: 1) Using a traditional (magnetic) motor to oscillate a one-dimensional (1D) transducer array which sweeps a planar image slice perpendicular to the image slice, forming a three-dimensional image. However, traditional (magnetic) motors contain ferromagnetic materials which are not compatible with MR machines. 2) A 2D matrix array transducer can be used to electronically steer the ultrasound beam over a volume. However, the additional electronics inside the probe handle that are required to operate a matrix array transducer poses a great challenge in the MR environment due to the need for a uniform magnetic field and sensitivity of both imaging systems to small electrical signals. Conversely, the present approach provides real-time three- and/or four-dimensional imaging using an MR compatible, hands-free electronic 4D ultrasound probe.

The present disclosure provides hands-free, real-time volumetric ultrasound imaging with MR compatibility for simultaneous MR and ultrasound imaging. Disclosed herein is an ultrasound probe for combined real-time three-dimensional ultrasound imaging with simultaneous magnetic resonance (MR) imaging. While the present disclosure is discussed in terms of radiation therapy, the MR-compatible ultrasound probe and the combination of simultaneous MR and ultrasound imaging may also be applied to other image-guided procedures such as proton therapy, biopsies, brachytherapy, surgery, and drug delivery. MR-compatibility, as discussed with reference to the disclosed ultrasound probe, refers an ultrasound probe that does not produce significant MR or ultrasound image artifacts during simultaneous operation. The MR-compatible ultrasound probe may contain a 2D matrix array and integrated beamforming electronics which are specially designed to minimize ferromagnetic content for MR compatibility. A low-profile, hands-free design of the MR-compatible ultrasound probe may allow the probe to be strapped to a patient so that ultrasound image acquisition may be achieved without needing a sonographer. A long probe cable (e.g., 6 m, 7m, 8m, 9m, 10m, and so forth) may connect the ultrasound probe in the MR room to a standard ultrasound system in a separate control room. The MR-compatible ultrasound probe and cable may be enclosed in an electromagnetic interference (EMI) shield which is continuous with a shield of the MR room to minimize ultrasound and MR system interference. Simultaneous ultrasound and MR imaging allows clinicians to combine the real-time capabilities of ultrasound with the soft-tissue image quality of MR for improved image guided radiation therapy (IgRT) at greatly reduced costs compared to combined MR-LINAC systems.

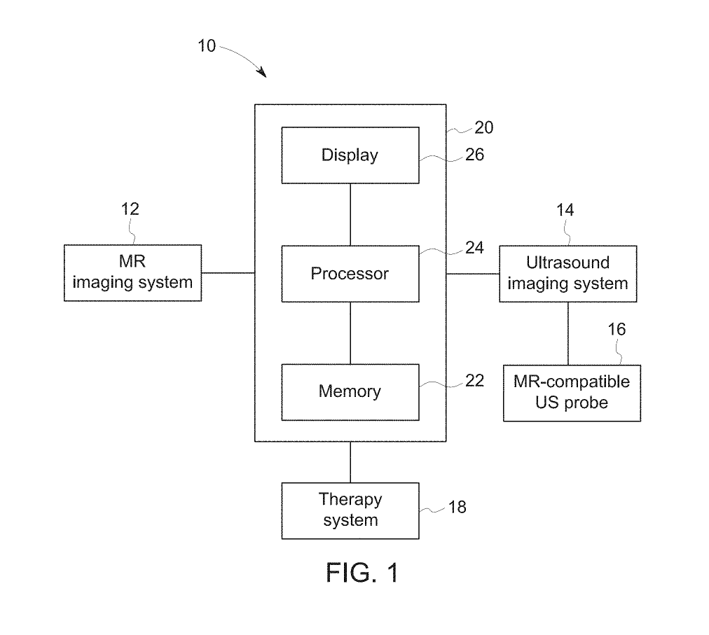

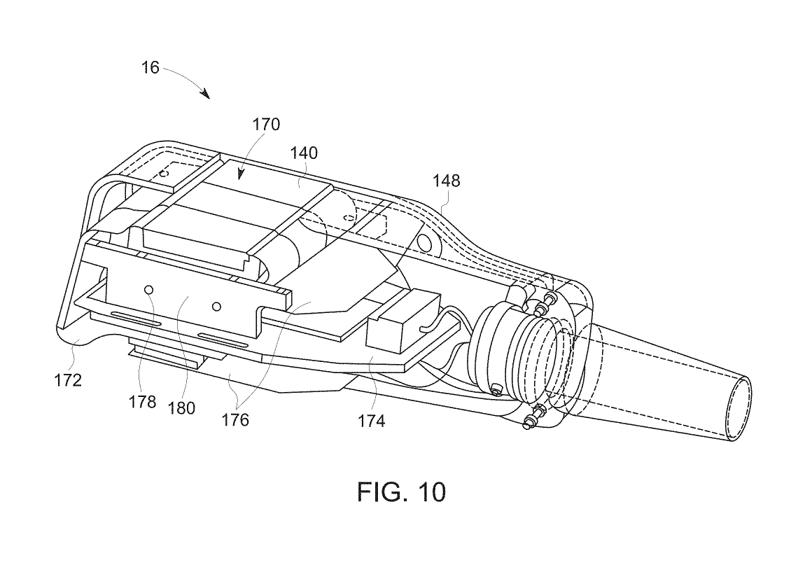

With the preceding comments in mind, FIG. 1 illustrates a schematic diagram of an embodiment of a combined MR and ultrasound imaging system 10 that may be used for non-invasive motion management of radiation therapy, or other therapy or surgical procedures, as described herein. The combined MR and ultrasound imaging system 10 includes a magnetic resonance (MR) imaging system 12 and an ultrasound imaging system 14. The ultrasound imaging system 14 may be communicatively coupled to a MR-compatible ultrasound probe 16. The MR-compatible ultrasound probe 16 may be an ultrasound probe configured for use in combination with the MR imaging system 12. As such, the MR-compatible ultrasound probe may contain low or no ferromagnetic material (e.g., iron, nickel, cobalt) content, as discussed in greater detail with reference to FIG. 10. The combined MR and ultrasound imaging system 10 may include a therapy system 18, such as a LINAC system used for radiation therapy. The therapy system 18 may be guided by images obtained via the MR imaging system 12 in combination with images obtained via the ultrasound imaging system 14 to help non-invasively manage motion of a target within a patient to improve accuracy of therapy from the therapy system 18.

The combined MR and ultrasound imaging system 10 may further include a controller 20 communicatively coupled to the other elements of the combined MR and ultrasound imaging system 10, including the MR imaging system 12, the ultrasound imaging system 14, and the therapy system 18. The controller 20 may include a memory 22 and a processor 24. In some embodiments, the memory 22 may include one or more tangible, non-transitory, computer-readable media that store instructions executable by the processor 24 and/or data to be processed by the processor 24. For example, the memory 22 may include random access memory (RAM), read only memory (ROM), rewritable non-volatile memory such as flash memory, hard drives, optical discs, and/or the like. Additionally, the processor 24 may include one or more general purpose microprocessors, one or more application specific processors (ASICs), one or more field programmable logic arrays (FPGAs), or any combination thereof. Further, the memory 22 may store instructions executable by the processor 24 to perform the methods described herein for the combined MR and ultrasound imaging system 10. Additionally, the memory 22 may store images obtained via the MR imaging system 12 and the ultrasound imaging system 14 and/or algorithms utilized by the processor 24 to help guide the therapy system 18 based on image inputs from the MR imaging system 12 and the ultrasound imaging system 14, as discussed in greater detail below. Further, the controller 20 may include a display 26 that may be used to display the images obtained by the MR imaging system 12 and the ultrasound imaging system 14.

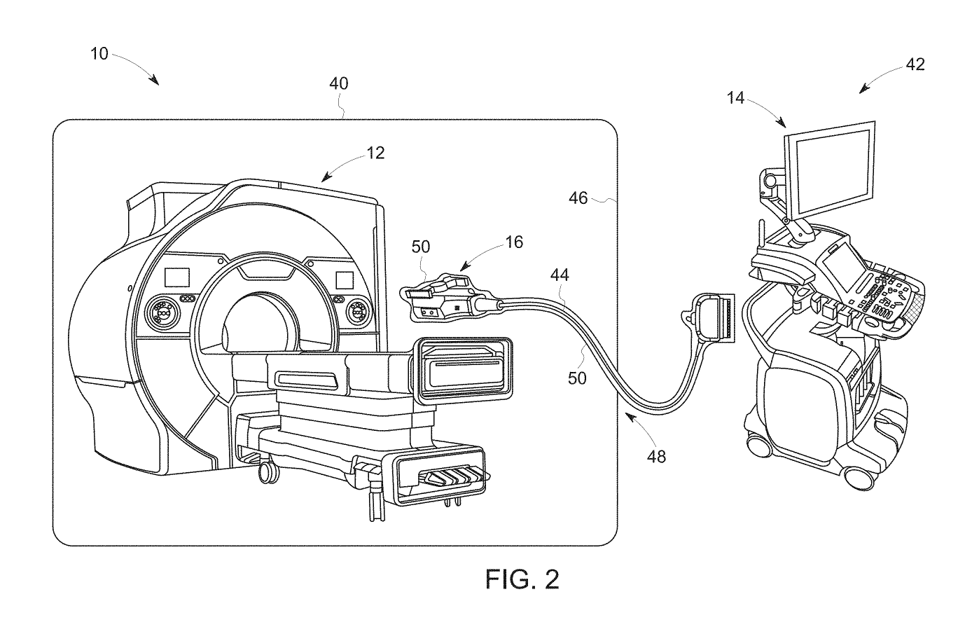

FIG. 2 illustrates an embodiment of an arrangement of the combined MR and ultrasound imaging system 10 having the ultrasound imaging system 14 outside of an MR room 40 containing the MR imaging system 12. In such an arrangement, the full ultrasound imaging system 14 is positioned within an ultrasound or control room 42, or other location outside of the MR room containing the MR imaging system 12. The MR-compatible ultrasound probe 16 is disposed within the MR room 40 and may be coupled to the ultrasound imaging system 14 via a long ultrasound probe cable 44 (e.g., longer than three meters). The relatively long ultrasound probe cable 44 does not significantly degrade the image quality of the ultrasound images obtained via the MR-compatible ultrasound probe 16 due to the presence of transmitter(s) and low-noise amplifier(s) in a handle of the MR-compatible ultrasound probe 16, impedance matching of the ultrasound probe cable 44, or a combination thereof. The ultrasound probe cable 44 may extend through a shielded wall 46 of the MR room 40 at a penetration location 48 and may couple to the MR-compatible ultrasound probe 16 within the MR room 40.

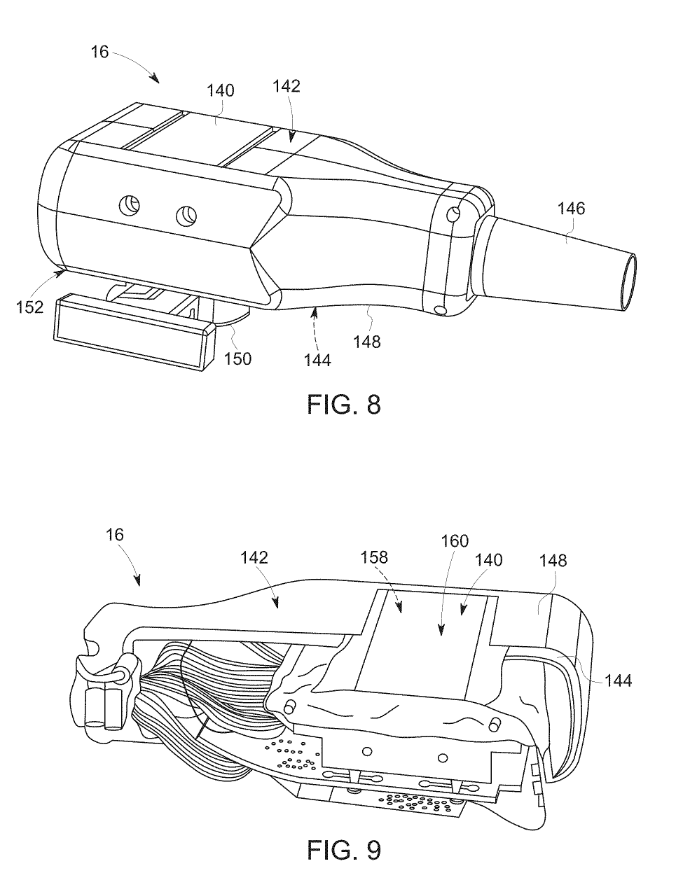

The ultrasound probe cable 44, as well as the MR-compatible ultrasound probe 16, may be enclosed in a shield 50 to provide full electromagnetic interference (EMI) shielding to minimize or prevent interference between the MR imaging system 12 and the ultrasound imaging system 14. Double shielding, of the MR compatible ultrasound probe 16 and the ultrasound probe cable 44, may allow for substantially reduced interference between MR image acquisition during simultaneous operation of the MR-compatible ultrasound probe 16, as well as substantially artifact-free operation of the MR-compatible ultrasound probe 16 within the MR-compatible ultrasound probe 16. The shield 50 may be an extension of the shield of the shielded wall 46 of the MR room 40. As the ultrasound probe cable 44 is passed through the shielded wall 46 of the MR room 40, the shield 50 may be electrically connected to the MR room shield 46, and may thus be grounded by the MR room shield 46. The ultrasound probe cable 44 and the MR-compatible ultrasound probe 16 may be physically and electrically shielded by the shield 50, which will be discussed in greater detail with reference to FIGS. 8, 9, 14A, and 14B.

Such an arrangement of the combined MR and ultrasound imaging system 10 may allow for use of a stock ultrasound system, without a need of modification of the ultrasound system or a specialized ultrasound system. Thus, the combined MR and ultrasound imaging system 10 may provide non-invasive motion management for therapies, as discussed in greater detail below, by combining real-time volumetric imaging capabilities of the ultrasound imaging system 14 and the MR-compatible ultrasound probe 16 with the increased soft tissue contrast and spatial resolution of the MR imaging system 12, while keeping costs for such increases relatively low. Additionally, the shielding of the ultrasound probe cable 44 and the MR-compatible ultrasound probe 16 via the shield 50 may provide MR-compatibility and minimize or prevent interference between the MR imaging system 12 and the MR-compatible ultrasound probe 16 and the ultrasound imaging system 14.

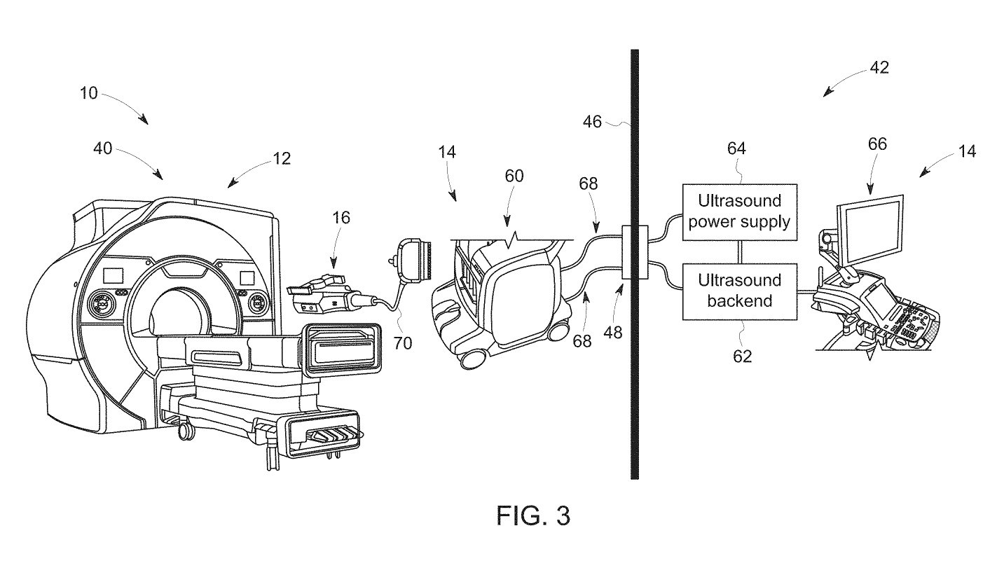

FIG. 3 illustrates an alternative embodiment of the combined MR and ultrasound imaging system 10 having a split ultrasound system arrangement. In such arrangements, the ultrasound imaging system 14 may be split into an MR-compatible ultrasound front end 60 and an ultrasound backend 62. The ultrasound backend and an ultrasound power supply 64 may be positioned within the ultrasound or control room 42 (e.g., ultrasound control room). The ultrasound power supply 64 and the ultrasound backend 62 may be separate components or may be housed together in a single unit 66, which may include a display or interface. The MR-compatible ultrasound front end 60 may be positioned within the MR room, along with the MR-compatible ultrasound probe 16. Power and digital communication lines 68 may pass through the shielded wall 46 of the MR room 40 at the penetration location 48 to communicatively couple the MR-compatible ultrasound front end 60 and the ultrasound backend 62 and the ultrasound power supply 64. Since the ultrasound imaging system 14 in the illustrated embodiment includes the MR-compatible ultrasound front end positioned within the MR room 40, a shorter ultrasound probe cable 70 (e.g., two meters to three meters) may be sufficient to couple the MR-compatible ultrasound probe 16 to the MR-compatible ultrasound front end 60. The relatively shorter ultrasound probe cable 70 may allow for use of MR-compatible ultrasound probes 16 that do not have transmitter(s) and/or low-noise amplifier(s) integrated into the handle of the MR-compatible ultrasound probe 16, as reducing the length of the ultrasound probe cable 70 and impedance matching of the ultrasound probe cable 70 may improve image quality of the images obtained via the MR-compatible ultrasound probe 16 as compared to a longer ultrasound probe cable.

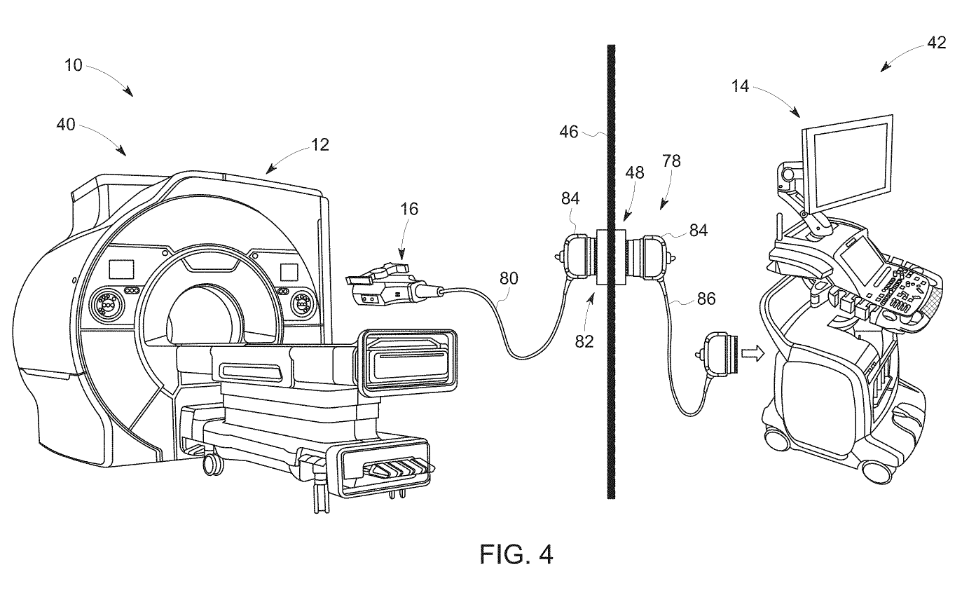

FIG. 4 illustrates an embodiment of a connection 78 between the MR-compatible ultrasound probe 16 via a relatively long shielded ultrasound probe cable 80 (e.g., long ultrasound probe cable 44 and shield 50 of FIG. 2) and the ultrasound imaging system 14 in arrangements of the combined MR and ultrasound imaging system 10 having only the MR-compatible ultrasound probe 16 positioned within the MR room 40 (e.g., arrangement shown in FIG. 2). The connection 78 may include a penetration (PEN) panel 82 with an electronics board that is positioned at the penetration location 48 through the shielded wall 46 of the MR room 40. The long shielded ultrasound cable 80 may couple to the MR-compatible ultrasound probe 16 at one end and may couple to a multi-pin connector 84 at the other end. The multi-pin connector 84 may couple the shielded ultrasound probe cable 80 to the PEN panel 82. The PEN panel 82 and the multi-pin connector 84 may form a continuous shield with the shield of the shielded ultrasound probe cable 80 and the shielded wall 46 of the MR room 40, and thus, may provide an approximately fully shielded connection through the shielded wall 46.

To connect the ultrasound imaging system 14 positioned outside of the MR room 40 to the PEN panel 82 and the MR-compatible ultrasound probe 16 within the MR room 40, a PEN-system cable 86 may couple to the PEN panel 82 through the shielded wall 46 of the MR room 40. The PEN-system cable 86 may couple to the PEN panel 82 via another multi-pin connector 84 on one end of PEN-system cable 86. The other end of the PEN-system cable 86 may couple to the ultrasound imaging system 14 via any suitable connection. In some embodiments, the PEN panel 82, the electronic board installed into the PEN panel 82, and/or one or both or the multi-pin connectors 84 may include passive and/or active electronic circuits such as filters, amplifiers, and digital communication repeaters, which may improve image quality and communication between the MR-compatible ultrasound probe 16 and the ultrasound imaging system 14, such as via tuning, amplifying, and filtering. The use of the PEN panel 82 and multi-pin connectors 84 as the connection 78 at the penetration location 48 through the shielded wall 46 of the MR room may provide approximately full shielding through the shielded wall 46 to minimize or prevent interference between the imaging systems. Further, the PEN panel 82 may include filters, amplifiers, and/or digital communication repeaters that may improve communication and image quality from the MR-compatible ultrasound probe 16 to the ultrasound imaging system 14.

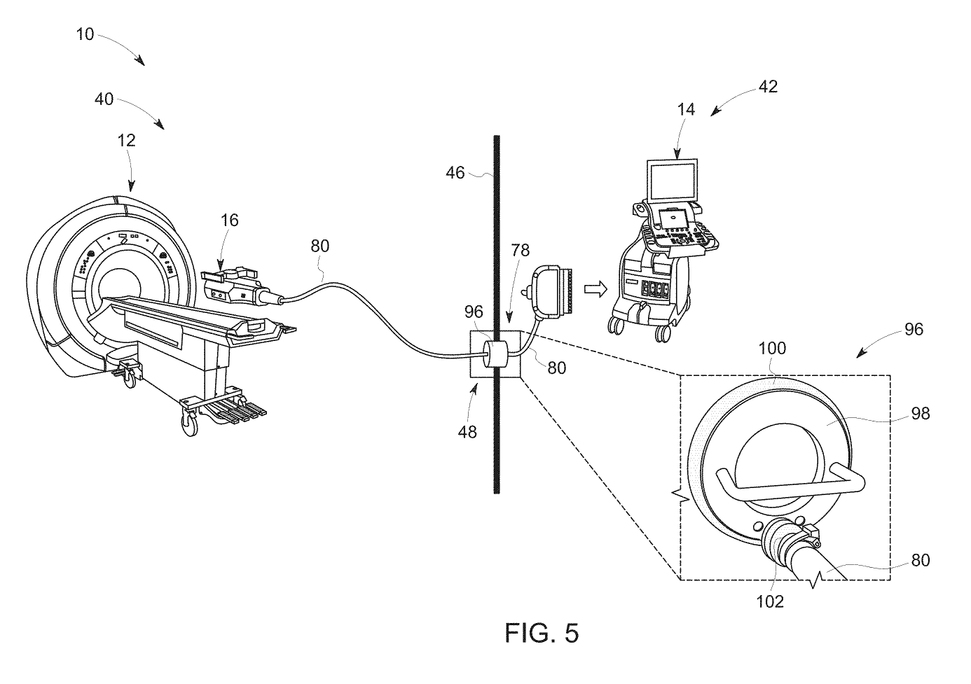

FIG. 5 illustrates an alternative embodiment the connection 78 between the MR-compatible ultrasound probe 16 via a relatively long shielded ultrasound probe cable 80 and the ultrasound imaging system 14 in arrangements of the combined MR and ultrasound imaging system 10 having only the MR-compatible ultrasound probe 16 positioned within the MR room 40 (e.g., arrangement shown in FIG. 2). In the illustrated embodiment, the connection 78 includes a waveguide 96 (e.g., tubular waveguide opening) through which the long shielded ultrasound cable 80 is passed through the shielded wall 46 of the MR room 40 at the penetration location 48. The long shielded ultrasound cable 80 may couple to the MR-compatible ultrasound probe 16 in the MR room at one end and may couple to the ultrasound imaging system 14 in the ultrasound room 42 via any suitable connection at the other end. To do so, the long shielded ultrasound cable 80 passes through the waveguide 96 in the shielded wall 46. The long shielded ultrasound probe cable 80 may be passed through the waveguide 96 and a conductive insert 98 and may couple to the ultrasound imaging system 14 in the ultrasound room 42 via any suitable connection.

The connection 78 may include the conductive insert 98 that may be positioned within the walls of the waveguide 96. The conductive insert 98 may be made from any suitable conductive material, such as aluminum. A gasket 100 may be disposed around the conductive insert 98 within the waveguide 96 to form an electrical connection between the conductive insert 98 and the waveguide 96. The gasket 100 may be an EMI gasket to provide EMI shielding of the ultrasound probe cable 80 as it passes through the shielded wall 46. The shielded probe cable 80 may pass through and couple to the conductive insert 98 via a gasket 102 to form an electrical connection between the shield of the shielded ultrasound probe cable 80 and the conductive insert 98 into the waveguide 96. The gasket 102 may be an EMI gasket to provide EMI shielding of the ultrasound probe cable 80 as it passes through the shielded wall 46. Thus, the long shielded ultrasound probe cable 80 may pass through an opening in the conductive insert 98 and may be physically and electrically coupled to the conductive insert 98 via the gasket 102. The conductive insert 98 may be inserted into the waveguide 96 at the penetration location 48 in the shielded wall 46. The gasket 100 may physically and electrically couple the conductive insert 98 to the shielded wall 46 of the MR room 40. Therefore, the long shielded ultrasound probe cable 80 may be electrically connected to and grounded by the MR room shield 46 via the electrical connections between the shield of the shielded ultrasound probe cable 80, the gasket 102, the conductive insert 98, and the gasket 100. The waveguide 96 and the conductive insert 98 may provide a shielded, low impedance, low inductance path for the shielded ultrasound probe cable 80 from MR-compatible ultrasound probe 16 in the MR room 40 to the ultrasound imaging system 14 in the ultrasound room 42.

Utilization of the combined MR and ultrasound imaging system 10 for providing and using virtual real-time MR images for motion management to guide radiation therapy, or other therapy, may consist of two stages: (1) a pre-treatment image acquisition stage; and (2) a treatment stage. The steps of the pre-treatment stage may occur at any time prior to the treatment state and may occur at a different location. For example, the pre-treatment stage may be conducted in the MR room 40 and the treatment stage may be performed in a radiation therapy room, other therapy room, or any suitable room for the treatment or procedure being performed.

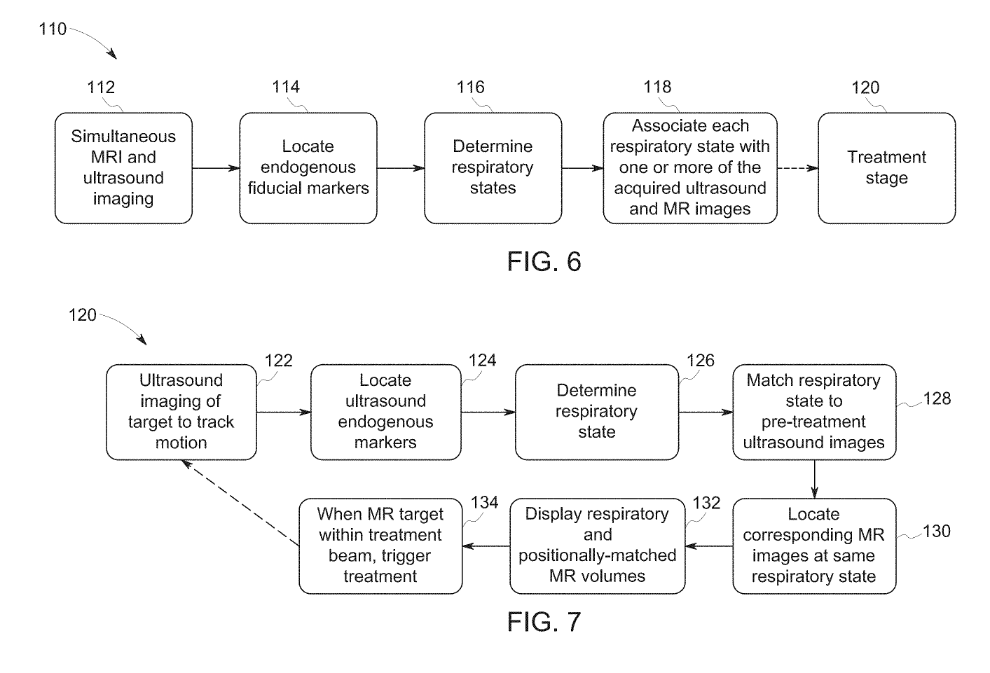

FIG. 6 illustrates a method 110 of the pre-treatment stage for providing virtual real-time MR images that may be used to guide radiation therapy in the treatment stage, discussed in greater detail with reference to FIG. 7. During the pre-treatment stage, at step 112, MR images and four-dimensional (4D) (e.g., real-three-dimensional) ultrasound images are simultaneously or nearly simultaneously acquired of the tumor or treatment target using the MR imaging system 12 and the MR-compatible ultrasound probe 16. The MR images and ultrasound images do not have to be completely aligned in time. If the images are not temporally aligned, techniques, such as temporal interpolation, may be used to substantially align or substantially link the images. Next, at step 114, one or more endogenous fiducial markers may be identified in the ultrasound images at each time point. For example, the endogenous fiducial markers may include blood vessels, structural anatomy of adjacent tissues, or the tumor or treatment target itself.

Next, at step 116, respiratory states at each time point of the ultrasound images corresponding to the respiratory motion of the patient are determined using positional or shape changes in the ultrasound images of the one or more endogenous fiducial markers identified at step 114. The respiratory states represent the possible respiratory states the patient may experience during the treatment procedure, for both the pre-treatment and treatment stages. For example, the respiratory states may include inhalation, exhalation, short-breath holds, irregular breaths, or any sub-state of a respiratory state. Next, at step 118, each determined respiratory state or sub-state is then associated with one or more of the acquired ultrasound and MR images. That is, the MR images corresponding to the ultrasound images at each time point may be resorted according to the determined respiratory states. A table or index of the determined respiratory states with their corresponding MR images may be created. Once the MR image index is created, these virtual real-time MR images may be used in the treatment stage, step 120 (e.g., method 120) to manage motion of the tumor or treatment target to help better guide the treatment to the treatment target.

FIG. 7 illustrates the method 120 of the treatment stage for utilizing the virtual real-time MR images to guide radiation therapy, or other therapy procedures. During the treatment stage, at step 122, ultrasound images (e.g., 4D ultrasound images, real-time 3D ultrasound images) of the tumor or treatment target are acquired in real-time to track the tumor or treatment target motion. Next, at step 124, the same endogenous one or more fiducial markers are identified and located in the ultrasound images. Next, at step 126, the patient's current respiratory state in the ultrasound images is determined by analysis of displacement of the one or more fiducial markers, and, at step 128, the respiratory state in the treatment stage ultrasound images is matched to the respiratory state in the pre-treatment ultrasound images. Once the respiratory state match is found, next, at step 130, the corresponding pre-treatment MR images that are indicative of the patient's current respiratory state are located using the index or table created in the method 110 of the pre-treatment stage. Thus, the pre-treatment MR images indicative of the patient's current respiratory state matched to real-time ultrasound images creates virtual real-time MR images of the tumor or treatment target.

The respiratory state matching steps 124, 128, and 130 may be represented by a single mathematical transfer function or separate mathematical transformation functions. For example, the mathematical transformation functions may represent a mapping of one respiratory state to another, one positional state of a deformable anatomical structure to another positional state, or a combination of both. A person of ordinary skill in the art should recognize that the mathematical transformation function may be any suitable geometric operation utilized with the observed anatomical markers in the ultrasound and MR images.

Next, at step 132, the MR images indicative of the patient's current respiratory state are displayed, allowing high resolution and contrast visualization of the tumor or treatment target motion to help guide the radiation or other therapy procedure. The MR images may be displayed to provide an accurate, real-time representation of the position of the tumor or treatment target and the surrounding anatomical details to guide the therapy procedure. However, a signal, such as a red dot, may be displayed if no MR image is available that corresponds to the current respiratory state of the patient.

Next, at step 134, when the tumor or target in the MR images indicative of the patient's current respiratory state is within the treatment line of the therapy system, the treatment may be triggered. For example, when the MR tumor or treatment target is within the LINAC beam, the LINAC beam is triggered to delivery guided radiation therapy to the tumor target. Therefore, the method 120 in combination with the pre-treatment method 110 may provide MR image guidance during the therapy procedure may be realized without a combined MR-treatment system (e.g., MR-LINAC system), which can minimize costs while providing a multi-modality imaging system which combines the real-time volumetric imaging capabilities of a 4D ultrasound probe with the soft tissue contrast and spatial resolution of MR imaging for non-invasive motion management of therapy procedures.

The pre-treatment method 110 for acquiring and providing the virtual real-time MR images and the treatment method 120 may be performed using the combined MR and ultrasound imaging system 10 and coupled therapy system 18. The processed and algorithms used in the methods 110 and 120, for example to identify fiducial markers, determine respiratory states, create the MR index or table, match ultrasound images and corresponding MR images, and trigger the treatment based on the real-time virtual MR images may be stored in the memory 22 and executed by the processor 24 of the controller 20 of the combined MR and ultrasound imaging system 10. In some embodiments, all or part of these processes may be performed and/or controlled by the controller 20 of the combined MR and ultrasound imaging system 10.

In order to perform the pre-treatment and treatment methods 110 and 120 to help manage motion and guide therapy procedures, the MR-compatible ultrasound probe 16 may be adapted to have particular form factors, such as a low-profile design and MR compatibility, as discussed in reference to FIGS. 8-10 and 13. FIG. 8 shows a perspective view of an embodiment of the MR-compatible ultrasound probe 16. The MR-compatible ultrasound probe 16 may be a real-time, three-dimensional (e.g., E4D) ultrasound probe that is low-profile, hands free, MR-compatible, and compatible with the therapy system 18 (e.g., LINAC system). The illustrated embodiment shows the MR-compatible ultrasound probe 16 having a transducer 140 on a patient-facing surface 142 of the MR-compatible ultrasound probe 16 and integrated beamforming electronics inside a probe housing 148. In some embodiments, the transducer 140 may be a 10,000+ element 2D array transducer. The internal beamforming electronics may reduce a signal count from 10,000+ 2D array elements of the transducer 140 to approximately two hundred channels connected to the ultrasound system via the shielded ultrasound probe cable 80. The shielded ultrasound probe cable 80 may be coupled to the MR-compatible ultrasound probe 16 via a cable connector 146 and a mechanical clamp within the probe housing 148. In one implementation, the MR-compatible ultrasound probe 16 may have 18,000 elements, provided as a 46.8 mm×21.5 mm 2D array transducer, and may include integrated beamforming electronics.

To provide shielding and MR-compatibility of the MR-compatible ultrasound probe 16 to minimize interference between the MR-compatible ultrasound probe 16, the MR imaging system 12, and the therapy system 18, the MR-compatible ultrasound probe 16 may be enclosed in an EMI shield 144. In some embodiments, the EMI shield 144 may be made from aluminum, and may also act as a heat spreader. The EMI shield 144 may be shaped such that it matches the shape of the housing 148 (e.g. plastic housing) of the MR-compatible ultrasound probe 16 to help maintain a low-profile of the MR-compatible ultrasound probe 16 and to increase heat transfer from the EMI shield 144 to the housing 148. Heat generated from the electrical components of the probe body may be spread over a larger area by the EMI shield 144, which also functions as a heat spreader. The EMI shield/heat spreader is in thermal contact with the outer housing 148 so that the heat is eventually dissipated to the ambient. The entirety of the electrical components of the MR-compatible ultrasound probe 16 are enclosed in the full EMI shield 144 to prevent unwanted interference between the MR-compatible ultrasound probe 16 and the MR imaging system 12. As previously discussed, the EMI shield 144 may be fully enclosed as an extension of the MR room shield 46. Additionally, to increase MR-compatibility of the MR-compatible ultrasound probe 16, components of the MR-compatible ultrasound probe 16 may be changed or chosen to have very low or no ferromagnetic material content for MR-compatibility, as discussed in greater detail with reference to FIG. 10. Additionally, the MR-compatible ultrasound probe 16 may be designed to minimize loops in electronic circuitry to avoid induced currents in the changing magnetic field.

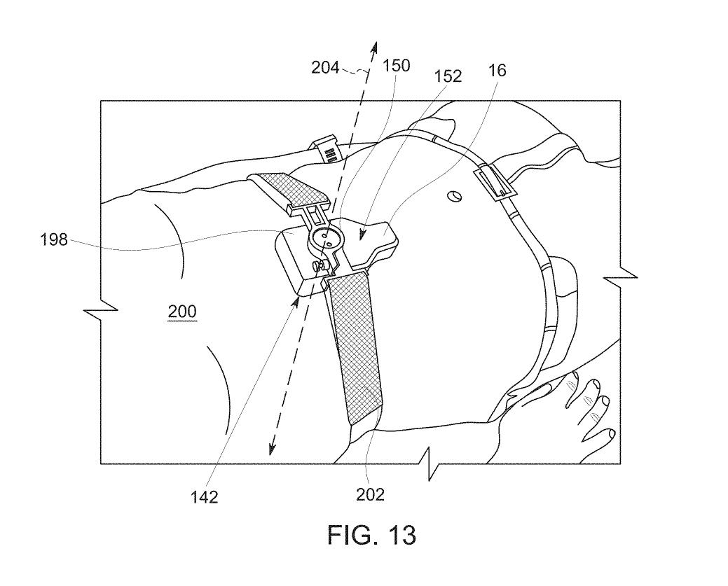

In operation, the MR-compatible ultrasound probe 16 may be fixed to the patient to help avoiding having a technician or sonographer holding the MR-compatible ultrasound probe 16 in place in the limited space between the patient and an inside wall of the MR imaging system 12 and during therapy procedures (e.g., radiation therapy procedures). To help enable the MR-compatible ultrasound probe 16 to be low-profile and hands-free, the MR-compatible ultrasound probe 16 may include a fastener 150, such as a hook and loop fastener or other suitable fastener, disposed on a non-transducer surface 152 of the MR-compatible ultrasound probe opposite the patient-facing surface 142. The fastener 150 may provide an attachment location for a strap to be secure, which may help the MR-compatible ultrasound probe remain stationary, as discussed in greater detail with reference to FIG. 13. Additionally, the fastener 150 may allow the MR-compatible ultrasound probe to be rotated to any orientation in order to acquire images of the tumor or treatment target.

FIG. 9 shows a cut-away view of an embodiment of the MR-compatible ultrasound probe 16. As previously discussed, the entire MR-compatible ultrasound probe 16 may be enclosed in the EMI shield 144 (e.g., aluminum shield), except for the transducer 140 (e.g., active acoustic aperture). In some embodiments, a face 158 of the transducer 140 on the patient-facing surface 142 may be covered or shielded by a thin foil 160 (e.g. 0.0122 mm thick aluminum foil). The thin foil 160 may be approximately 10-15 microns thick, and may provide electrical shielding of the transducer 140 to help minimize interference between the MR-compatible ultrasound probe 16, the MR imaging system 12, and the therapy system 18, while having a negligible impact on the acoustic performance of the transducer 140. Therefore, the entire MR-compatible ultrasound probe 16 may be enclosed and electrically shielded by the EMI shield 144 and the thin foil 160, and the EMI shield may be surrounded by the housing 148.

As previously mentioned, to provide and/or increase MR-compatibility and compatibility with the therapy system 18 of the MR-compatible ultrasound probe 16, components of the MR-compatible ultrasound probe 16 may be changed or chosen to have very low or no ferromagnetic material content. Ferromagnetic materials may cause artifacts in the MR images. FIG. 10 shows a cross-sectional side view of an embodiment of the MR-compatible ultrasound probe 16 showing examples of particular components of the MR-compatible ultrasound probe 16 that may be changed or chosen so has to have very low or no ferromagnetic material content. Components of the MR-compatible ultrasound probe 16 that may typically contain ferromagnetic material may be made or replaced with materials having very low or no ferromagnetic content. Elements of an acoustic stack 170 of the transducer 140 may be changed for MR-compatibility. For example, ferromagnetic content of an interface layer may be reduced and an acoustic backing may be replaced with non-magnetic filled foam backing. Alternative metallization may be used for components that need metallization, such as an outer matching layer of the acoustic stack 170. A titanium tungsten combination (TiW), or other suitable non-ferromagnetic material, may be used to reduce or eliminate nickel (Ni), which is ferromagnetic, in ground metallization of the outer matching layer.

Additionally, materials for a flex interconnect 172 and an electronics board 174 of the MR-compatible ultrasound probe 16 may be changed to non-ferromagnetic passive components and connectors. Further, non-ferromagnetic connectors and a direct solder coax may be used for one or more system channel boards 176. Additionally, any mechanical fasteners used within the MR-compatible ultrasound probe 16, such as screws 178 used to fasten a heat sink 180 to the MR-compatible ultrasound probe 16, may be non-ferromagnetic screws, e.g., brass screws. Other components of the MR-compatible ultrasound probe 16 may be changed to help increase the MR-compatibility of the MR-compatible ultrasound probe 16.

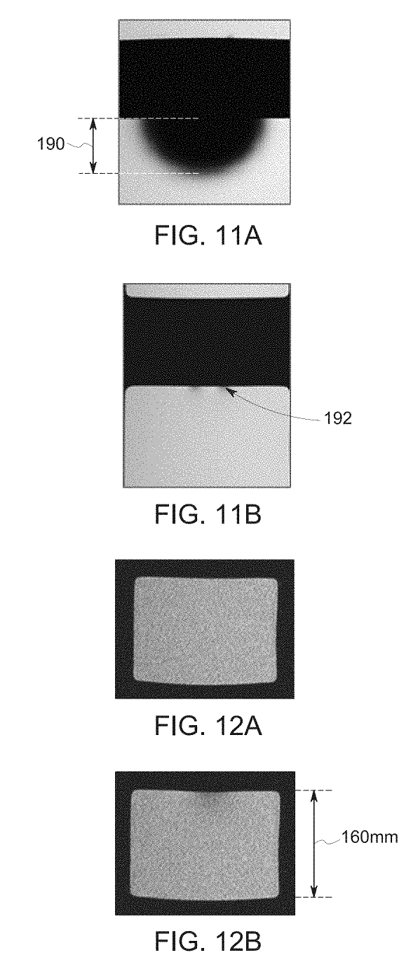

To illustrate the increase in MR image quality that may be provided by reducing ferromagnetic materials content from the MR-compatible ultrasound probe 16 to increase MR-compatibility, FIGS. 11A and 11B illustrate example MR-compatibility test images for the acoustic stack 170 of the MR-compatible ultrasound probe 16. FIG. 11A shows an MR image obtained when conventional acoustic stack material was placed on an MR imaging phantom. The conventional acoustic stack material, containing ferromagnetic material, resulted in a large artifact 190 that measured several centimeters in depth due to the ferromagnetic content. FIG. 11B shows an MR image obtained using alternative acoustic stack material containing significantly less ferromagnetic material, which may be substituted for the conventional material in the MR-compatible ultrasound probe 16. The alternative acoustic stack material result in a greatly reduced MR artifact 192. Reducing or substantially eliminating any ferromagnetic materials from the MR-compatible ultrasound probe 16 may reduce the appearance of artifacts in the MR images and increase MR-compatibility of the MR-compatible ultrasound probe 16.

Along the same lines, FIGS. 12A and 12B illustrate example MR-compatibility test images for the whole MR-compatible ultrasound probe 16. For comparison, FIG. 12A shows an MR image obtained of a phantom without a probe present. FIG. 12B shows the same MR image phantom with the MR-compatible ultrasound probe 16 placed on the topside. As the MR-compatible ultrasound probe 16 is designed to contain minimal ferromagnetic materials, any MR artifact due to the presence of the MR-compatible ultrasound probe 16 is minimal.

FIG. 13 illustrates a perspective view of an embodiment of the MR-compatible ultrasound probe 16 positioned on a patient 200. For illustrative purposes, the ultrasound cable is not shown. In operation, there may be limited space available between the patient 200 and an inside wall of the MR imaging system 12. Therefore, the MR-compatible ultrasound probe 16 may have a low-profile design or form factor. In one embodiment, a body 198 of the MR-compatible ultrasound probe 16 may have dimensions such as 116 mm length, 65 mm height, and 36 mm depth. A relatively shallow depth may allow the MR-compatible ultrasound probe 16 to fit within the limited space of the MR imaging system 12.

Further, the MR-compatible ultrasound probe 16 may be fixed to the patient 200 so that hands-free images of the tumor or treatment target may be obtained without needing a sonographer. The illustrated embodiment shows the low-profile, hands-free design of the MR-compatible ultrasound probe 16. To acquire images, the MR-compatible ultrasound probe 16 may be positioned against the patient 200 with the patient-facing surface 142 having the covered transducer 140 facing toward the patient 200. As such, the fastener 150 disposed on the non-transducer surface 152 is positioned away from the patient 200. The fastener 150 may serve as a connection location for a strap 202, or other device, which allows the MR-compatible ultrasound probe 16 to remain stationary against the patient 200 so that volumetric images are acquired without needing a sonographer. In some embodiments, the fastener 150 may further allow for rotation of the MR-compatible ultrasound probe 16 about a central axis 204 extending from through the patient-facing surface 142 and the non-transducer surface 152. Such rotation may allow the MR-compatible ultrasound probe 16 to be oriented in a position to accurately image the tumor or treatment target while the strap 202 remains in place around the patient 200.

Rotation of the MR-compatible ultrasound probe 16 about the central axis 204 may be by manual rotation, for example. In some embodiments, the MR-compatible ultrasound probe 16 may include a non-magnetic motor communicatively coupled to the controller 20, a control system of the ultrasound imaging system 14, or any other suitable controller. The motor may be disposed within the body 198 of the MR-compatible ultrasound probe 16, the fastener 150, or any other suitable position to control the orientation of the MR-compatible ultrasound probe 16 about the central axis 204. As such, in some embodiments, rotation of the MR-compatible ultrasound probe 16 may be electronically steerable about the central axis 204.

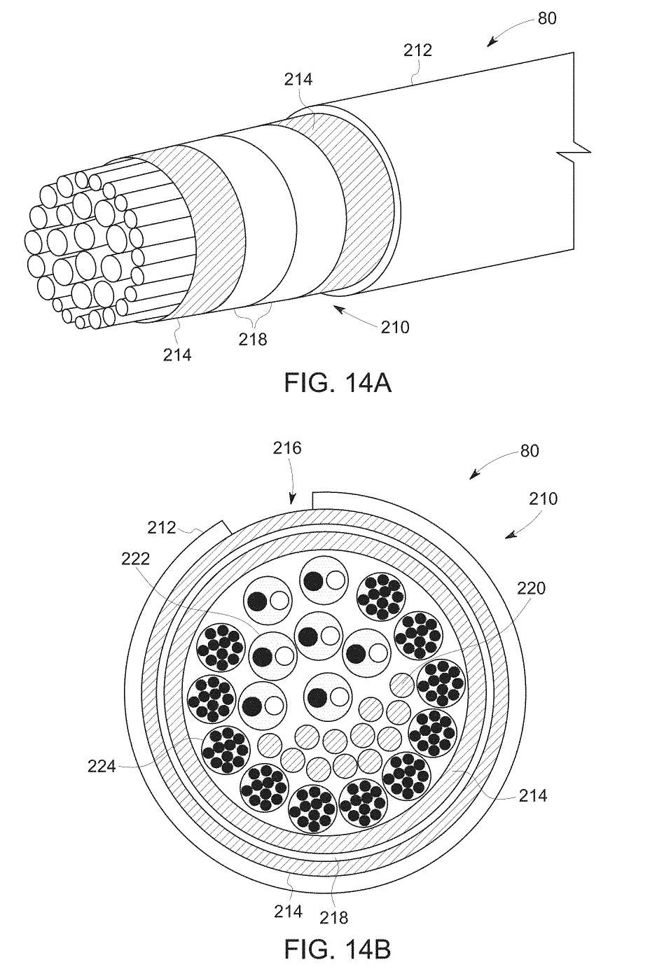

FIGS. 14A and 14B illustrate a shield 210 of the shielded ultrasound probe cable 80. As previously discussed, the shield 210 may be an extension of the MR room shield 46 and may provide full EMI shielding to the shielded ultrasound probe cable 80. The shield 210 of the shielded ultrasound probe cable 80 may contain multiple layers of overall shielding to help minimize EMI interactions between the ultrasound imaging system 14, including the MR-compatible ultrasound probe 16, and the MR imaging system 12. The shield 210 may be surrounded by an outer cable jacket 212 that may be made from an insulative material, such as a flexible polymer. Below the outer cable jacket 212 there may be an overall shield layer 214 that may be made from aluminized polyester, aluminized mylar, or other conductive wrap material. The overall shield layer 214 may be formed from wrapped foils, braided strands, or a similar composition of the conductive wrap material. In some embodiments, the outer cable jacket 212 may have a window 216 or space in which a portion of the outer cable jacket 212 is missing, exposing the conductive overall shield 214. Exposure of the conductive overall shield 214 via the window 216 may allow the overall shield 214 to be electrically accessed to electrically couple the shielded ultrasound probe cable 80 to the MR room shield 46, for example, via the conductive insert 98 and the waveguide 96, as discussed in reference to FIG. 5. Below the overall shield 214 may be one or more wire braid layers 218, which may each have approximately 95% coverage. Below the one or more wire braid layers 218, the shield 210 may include another overall shield 214 layer, such that the one or more wire braid layers 218 are positioned between two overall shield 214 layers. Within the multiple layers of the shield 210, the shielded ultrasound probe cable 80 may contain multiple bundle types, such as stranded wires 220, shielded, twisted pair 222, and coaxial cables 224, which may include individual shields. Within the shielded ultrasound probe cable 80, additional shielding for sensitive signals may be achieved by the use of the coaxial cables 224 and the shielded, twisted pair 222 as is common in the ultrasound industry.

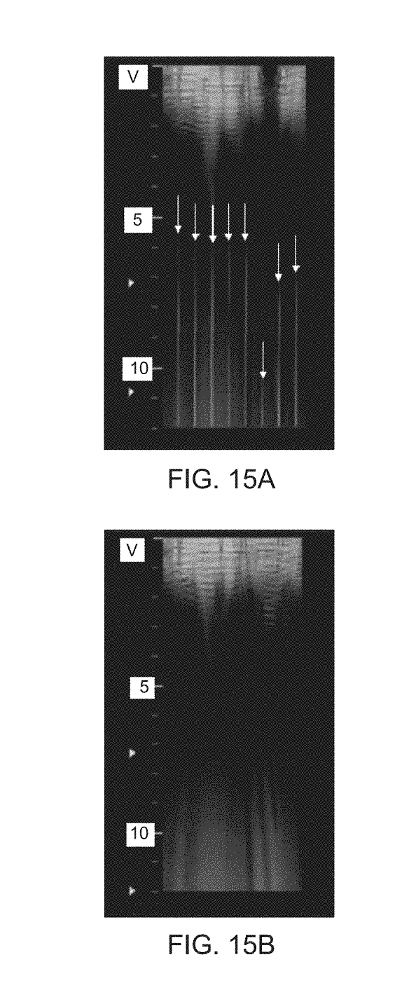

FIGS. 15A and 15B show ultrasound images demonstrating the effect of shielding the MR compatible ultrasound probe 16 and the shielded ultrasound probe cable 80 to minimize electromagnetic interference (EMI) between the MR imaging system 12 and the MR-compatible ultrasound probe 16 and the ultrasound imaging system 14. FIG. 15A shows artifacts (pointed out by the arrows in the image) in the ultrasound image due to MR radiofrequency transmit being picked up by an inadequately shielded probe and cable. However, FIG. 15B shows an ultrasound image where the artifacts in FIG. 15A are absent due to full EMI shielding of the MR-compatible ultrasound probe 16 and the shielded ultrasound probe cable 80 via the EMI shield 144, the thin foil 160 covering the transducer 140, and the shield layers 214 and 218.

Technical effects of the present disclosure include providing a low-profile, hands-free, MR-compatible real-time three-dimensional (e4D) ultrasound imaging probe for real-time volumetric ultrasound imaging with MR compatibility for simultaneous MR and ultrasound imaging. The MR-compatible ultrasound probe allows for acquisition of simultaneous volumetric ultrasound and MR images. The MR-compatible ultrasound probe may allow for use of a multi-modality imaging system which combines the real-time volumetric imaging capabilities of the MR-compatible ultrasound probe with the soft tissue contrast and spatial resolution of MR imaging for non-invasive motion management of radiation or other therapy. The low-profile, hands-free design of the MR-compatible ultrasound probe allows for volumetric ultrasound imaging without requiring a sonographer. This may free resources, and also allow for the use of ultrasound in radiation environments without the use of a sonographer. The MR-compatible ultrasound probe may contain components which are specially designed or changed to minimize ferromagnetic content to increase MR-compatibility. The MR-compatible ultrasound probe, shielded ultrasound probe cable, and connector have full EMI shielding that effectively isolates the ultrasound and MIR imaging systems so that there is negligible electrical interference between the ultrasound and MR imaging systems.

Use of a long shielded ultrasound probe cable may allow the MR-compatible ultrasound probe to be connected to a standard ultrasound system in a separate control room. Unlike conventional ultrasound probes, the image quality may not substantially degraded by the long cable due to the presence of transmitters and a low-noise amplifier integrated in the MR-compatible ultrasound probe handle electronics, impedance matching of the cable, or a combination thereof. Additional electronics such as filters, amplifiers, digital communication circuits may reside in the connectors and/or electronics boards between the MR-compatible ultrasound probe and the ultrasound system. The MR-compatible ultrasound probe may be fitted to standard MR suites, which may provide a low-cost alternative to the combined imaging and therapy systems. An alternative embodiment provides a split ultrasound system having an MR-compatible front end, and a power supply, backend, and user interface in a separate control room which allows the ultrasound probe cable to remain at a shorter length. This configuration is useful for MR-compatible ultrasound probes that do not have electronics such as transmitters and low noise amplifiers integrated in the probe handle.

This written description uses examples as part of the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An ultrasound probe configured for use in a multi-modality imaging system, comprising:
a body comprising one or more electrical components of the ultrasound probe;
an outermost housing enclosing the ultrasound probe;
an electromagnetic interference (EMI) shield surrounding the body and disposed between the body and the housing, wherein the EMI shield is configured to reduce interference between the ultrasound probe and one or more different imaging systems of the multi-modality imaging system;
a transducer disposed on a patient-facing surface of the ultrasound probe; and
a cable coupled to the body and configured to communicatively couple the ultrasound probe to an ultrasound imaging system of the multi-modality imaging system;
wherein the ultrasound probe comprises substantially non-ferromagnetic material.

2. The ultrasound probe of claim 1, wherein the cable comprises a second EMI shield enclosing the cable.

3. The ultrasound probe of claim 2, wherein the EMI shield comprises aluminum, and wherein the EMI shield is electrically coupled to the second EMI shield.

4. The ultrasound probe of claim 2, comprising a thin foil covering a face of the transducer, wherein the EMI shield, the second EMI shield, and the thin foil provide approximately full EMI shielding of the body, the transducer, and the cable of the ultrasound probe.

5. The ultrasound probe of claim 2, wherein the cable comprises an insulative outer layer surrounding the second EMI shield, and wherein the second EMI shield comprises a plurality of layers comprising one or more conductive wrap layers, one or more wire braid layers, or a combination thereof.

6. The ultrasound probe of claim 1, comprising one or more low noise amplifiers disposed within the body, wherein the cable has a length greater than three meters, and wherein visible image quality of the images obtained by the ultrasound probe is not significantly reduced by the length of the cable due to the presence of the one or more low noise amplifiers.

7. The ultrasound probe of claim 1, comprising a fastener disposed on a non-transducer surface of the ultrasound probe, wherein the fastener is configured to hold the ultrasound probe in place relative to a patient, and a strap coupled to the fastener and configured to hold the ultrasound probe in place against the patient.

8. The ultrasound probe of claim 7, wherein the fastener comprises a rotatable fastener rotatable about a central axis of the ultrasound probe, wherein rotation about the central axis allows an orientation of the ultrasound probe about the axis to be changed while the ultrasound probe is held against the patient via the strap.

9. The ultrasound probe of claim 8, wherein the EMI shield is configured to contact the housing such that heat may be transferred from the body to the EMI shield to the housing.

10. The ultrasound probe of claim 1, wherein the ultrasound probe is configured to be coupled to a stock ultrasound imaging system via the cable.

11. A multi-modality imaging system, comprising:
an ultrasound imaging system;
a magnetic resonance (MR) imaging system, wherein the MR imaging system is positioned within a shielded MR room comprising an MR room shield;
an MR-compatible ultrasound probe coupled to the ultrasound imaging system and configured to acquire ultrasound images while the MR-compatible ultrasound probe is positioned within the shielded MR room, wherein all or part of the ultrasound imaging system is positioned outside of the shielded MR room; and
a shielded ultrasound probe cable coupled to the MR-compatible ultrasound probe at a first end and coupled to the ultrasound system at a second end.

12. The multi-modality imaging system of claim 11, wherein the ultrasound imaging system comprises a stock ultrasound imaging system, and wherein all of the ultrasound imaging system is positioned outside of the shielded MR room.

13. The multi-modality imaging system of claim 11, wherein the ultrasound imaging system comprises a split ultrasound system comprising an MR-compatible front end configured to be positioned within the shielded MR room, an ultrasound backend, and an ultrasound power source, wherein the ultrasound backend and the ultrasound power source are configured to be positioned outside of the shielded MR room.

14. The multi-modality imaging system of claim 11, wherein the shielded ultrasound probe cable comprises a first EMI shield enclosing the shielded ultrasound probe cable, wherein the shielded ultrasound probe cable passes through the MR room shield at a penetration location, wherein the penetration location comprises one of a penetration (PEN) panel or a waveguide comprising a conductive insert, wherein the first EMI shield of the shielded ultrasound probe cable is electrically coupled to the MR room shield at the penetration location.

15. The multi-modality imaging system of claim 14, wherein the MR-compatible ultrasound probe comprises a second EMI shield, wherein the second EMI shield approximately fully encloses a body of the MR-compatible ultrasound probe, and wherein the first EMI shield and the second EMI shield are electrically coupled.

16. The multi-modality imaging system of claim 14, comprising a PEN-system cable, wherein the penetration location comprises a PEN panel, wherein the PEN-system cable is configured to couple the PEN panel to the ultrasound system, wherein the shielded ultrasound probe cable is configured to couple the MR-compatible probe to the PEN panel, and wherein the PEN panel comprises passive electronic components, active electronic components, or a combination thereof configured to substantially minimize any image quality loss.

17. A method, comprising:
positioning one or more electrical components of an ultrasound probe within a body;
surrounding the body with an electromagnetic interference (EMI) shield, wherein the EMI shield is configured to reduce interference between the ultrasound probe and one or more different imaging systems;
enclosing the body and the EMI shield within a housing, wherein the EMI shield is disposed between the body and the housing, and wherein the EMI shield contacts the housing;
disposing a transducer on a patient-facing surface of the ultrasound probe, wherein the transducer comprises substantially non-ferromagnetic materials; and
coupling a cable to the body, wherein the cable is configured to communicatively couple the ultrasound probe to an ultrasound imaging system.

18. The method of claim 17, comprising surrounding the cable with a second EMI shield and electrically coupling the EMI shield surrounding the ultrasound probe to the second EMI shield enclosing the cable.

19. The method of claim 18, comprising covering a face of the transducer with a thin foil, wherein the EMI shield, the second EMI shield, and the thin foil are configured to provide approximately full EMI shielding of the body, the transducer, and the cable of the ultrasound probe.

20. The method of claim 17, wherein the one or more electrical components comprise substantially non-ferromagnetic materials.
